(19) United States
(12) Patent Application Publication
(10) Pub. No.: US 2021/0219892 A1
(43) Pub. Date: Jul. 22, 2021

(54) DIGITAL BIOMARKERS FOR ASSESSING SCHIZOPHRENIA OR AN AUTISM SPECTRUM DISORDER

(71) Applicant: Hoffmann-La Roche Inc., Little Falls, NJ (US)

(72) Inventors: Christian Gossens, Basel (CH); Timothy Kilchenmann, Basel (CH); Michael Lindemann, Basel (CH)

(21) Appl. No.: 17/221,449

(22) Filed: Apr. 2, 2021

Related U.S. Application Data
(63) Continuation of application No. PCT/EP2019/076972, filed on Oct. 4, 2019.

(30) Foreign Application Priority Data
Oct. 5, 2018 (EP) 18 198 954.2

(51) Int. Cl. A61B 5/16; A61B 5/00; A61B 5/11; G16H 20/70; G16H 50/20; G16H 40/67

(52) U.S. Cl. CPC A61B 5/165; A61B 5/6898; A61B 5/1118; A61B 5/4842; A61B 5/168; A61B 5/0022; A61B 2560/0242; G16H 50/20; G16H 40/67; A61B 2562/0219; A61B 2562/0223; A61B 2562/0257; A61B 2562/0247; G16H 20/70

(57) ABSTRACT

The present disclosure relates to the field of schizophrenia or an autism spectrum disorder (“ASD”) diagnostics and disease management. Specifically, the present disclosure teaches a method of assessing schizophrenia or ASD in a subject in which a subject's usage data for a mobile device is collected over a first predefined time window. A usage behavior parameter is determined from the usage data, and the determined usage behavior parameter is compared to a reference. From the comparison it may be determined whether the schizophrenia or ASD in the subject is improving, persisting or worsening. A system including a mobile device having sensors recording usage data and a remote device operatively linked to the mobile device is also disclosed.

### RELATED APPLICATIONS

This application is a continuation of PCT/EP2019/076972, filed Oct. 4, 2019, which claims priority to EP 18198954.2, filed Oct. 5, 2018, the entire disclosures of both of which are hereby incorporated herein by reference.

### BACKGROUND

The present disclosure relates to the field of schizophrenia or an autism spectrum disorder diagnostics and disease management. Specifically, it relates to a method assessing schizophrenia or an autism spectrum disorder in a subject comprising the steps of determining at least one usage behavior parameter from a dataset comprising usage data for a mobile device within a first predefined time window wherein said mobile device has been used by the subject and comparing the determined at least one usage behavior parameter to a reference, whereby schizophrenia or an autism spectrum disorder will be assessed. The present disclosure also relates to a mobile device comprising a processor, at least one sensor recording usage data and a database as well as software which is tangibly embedded to said device and, when running on said device, carries out the aforementioned method. Also contemplated by the disclosure is a system comprising a mobile device comprising at least one sensor recording usage data and a remote device comprising a processor and a database as well as software which is tangibly embedded to said device and, when running on said device, carries out the aforementioned method, wherein said mobile device and said remote device are operatively linked to each other. Also, the disclosure relates to the use of the mobile device or the system for assessing schizophrenia by analyzing a dataset comprising usage data for a mobile device within a first predefined time window wherein said mobile device has been used by the subject.

Schizophrenia is a mental disease which requires great medical and financial efforts to be handled properly. Schizophrenia results in a decrease in life expectancy of between 10 to 25 years. The disease and its causes are still poorly understood, in particular, there is an ongoing discussion of causes such as obesity, poor diet, sedentary lifestyles, and smoking, as well as antipsychotic medications or drug abuse (cannabis) may also increase the risk. Risk factors for schizophrenia include gender, intelligence as well as the existence of other mental disease such as depression.

Approximately 75% of people with schizophrenia have ongoing disabilities with relapses. Moreover, about 16 million people globally are suspected to have moderate or a severe disability from the condition. The average suicide rate is higher in patients with schizophrenia. Some people recover completely and others at least integrate well in society, however, unemployment is an issue. Most people with schizophrenia are able to live independently with community support. In general, outcomes for schizophrenia appear to be better in the developed countries.

Conventional diagnostics for schizophrenia include self-reported experiences as well as assessment by mental health professionals. Various criteria have been established by the American Psychiatric Association released the fifth edition of the Diagnostic and Statistical Manual of Mental Disorders (DSM 5) or the World Health Organization's International Statistical Classification of Diseases and Related Health Problems (ICD-10). For diagnosing schizophrenia according to DSM 5, two criteria have to be met over a period of at least one month, with a significant impact on social or occupational functioning for at least six months. The person had to be suffering from delusions, hallucinations, or disorganized speech. The second symptom could be negative symptoms, or severely disorganized or catatonic behavior. The ICD-10 criteria put more emphasis on the so-called Schneiderian first-rank symptoms. In practice, agreement between the two systems is, however, high.

Various subtypes of schizophrenia exist: paranoid type, disorganized type, catatonic type, undifferentiated type, residual type, post-schizophrenic depression type, simple-schizophrenia type, other-schizophrenia type (see DSM 5 or ICD-10).

Differential diagnosis of schizophrenia may sometimes be difficult. In particular, several other mental diseases may be accompanied by similar symptoms, e.g., bipolar disorder, borderline personality disorder, drug intoxication, drug-induced psychosis, social anxiety disorder, avoidant personality disorder and schizotypal personality disorder. Moreover, neurological or general disease may result in similar symptoms, in particular, metabolic disturbance, systemic infection, syphilis, AIDS dementia complex, epilepsy, limbic encephalitis, brain lesions, stroke, multiple sclerosis, hyperthyroidism, hypothyroidism, and dementias such as Alzheimer's disease, Huntington's disease, frontotemporal dementia, and the Lewy body dementias.

The so-called Positive and Negative Syndrome Scale (PANSS) is a medical scale used for measuring symptom severity of patients with schizophrenia. Alternatively, the Brief Negative Symptom Scale (BNSS) may be applied. Both scales are based on interviews carried out by mental health specialist and require great experience of the interviewer in order to give comparable assessments.

There have been some reports on using smart phone data for digitally phenotyping patients suffering from schizophrenia and for monitoring them using a smartphone as a detector and a remote database system for evaluation (Torous 2016; Wang 2016).

Autism spectrum disorders are neurodevelopmental disorders including classical autism and related medical conditions. Autism spectrum disorders appear to have a prevalence of about 6 among 1,000 people. The rates appear to be consistent among different cultural and ethnic backgrounds. However, males appear to be affected more often than females. Typical symptoms include problems in social communication and social interaction, and restricted, repetitive patterns of behavior, interests or activities. Symptoms are usually recognized between one and two years of age. Long-term issues may include difficulties in creating and keeping relationships, maintaining a job, and performing daily tasks. The DSM 5 recognizes autism, Asperger syndrome, pervasive developmental disorder not otherwise specified (PDD-NOS), and childhood disintegrative disorder as disorders falling into the group of autism spectrum disorders. Genetic reasons as well as environmental influences are discussed as potential risk factors.

Nevertheless, there is a need for reliable measures for assessing schizophrenia and/or autism spectrum disorders in affected patients.

### SUMMARY

The technical problem underlying this disclosure may be seen in the provision of means and methods complying with the aforementioned needs. The technical problem is addressed by the embodiments described herein below.

The present disclosure relates to a method assessing schizophrenia or an autism spectrum disorder in a subject comprising the steps of:
a) determining at least one usage behavior parameter from a dataset comprising usage data for a mobile device within a first predefined time window wherein said mobile device has been used by the subject; and
b) comparing the determined at least one usage behavior parameter to a reference, whereby schizophrenia or an autism spectrum disorder will be assessed.

Typically, the method further comprises the step of (c) determining an improvement, persistency or worsening of the negative symptoms associated with schizophrenia or autism spectrum disorders in a subject based on the comparison carried out in step (b).

In some embodiments, the method may also comprise prior to step (a) the step of obtaining from the subject using a mobile device a dataset of usage data for a mobile device within a first predefined time window. However, typically the method is an ex vivo method carried out on an existing dataset comprising usage data for a mobile device within a first predefined time window which does not require any physical interaction with the subject.

The method as referred to in accordance with the present disclosure includes a method which essentially consists of the aforementioned steps or a method which may include additional steps.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, notwithstanding the fact that the respective feature or element may be present once or more than once.

Further, as used in the following, the terms “particularly”, “more particularly”, “specifically”, “more specifically”, “typically”, and “more typically” or similar terms are used in conjunction with additional/alternative features, without restricting alternative possibilities. Thus, features introduced by these terms are additional/alternative features and are not intended to restrict the scope of the claims in any way. The disclosure may, as the skilled person will recognize, be performed by using alternative features. Similarly, features introduced by “in an embodiment of the invention” or similar expressions are intended to be additional/alternative features, without any restriction regarding alternative embodiments of the invention, without any restrictions regarding the scope of the invention and without any restriction regarding the possibility of combining the features introduced in such way with other additional/alternative or non-additional/alternative features of the invention.

The method may be carried out on the mobile device by the subject once the dataset of comprising usage data for a mobile device within a first predefined time window has been acquired. Typically, the mobile device and the device acquiring the dataset may be physically identical, i.e., the same device. Such a mobile device shall have a data acquisition unit which typically comprises means for data acquisition, i.e., means which detect or measure either quantitatively or qualitatively physical parameters and transform them into electronic signals transmitted to the evaluation unit in the mobile device used for carrying out the method according to the disclosure. The data acquisition unit comprises means for data acquisition, i.e., means which detect or measure either quantitatively or qualitatively physical parameters and transform them into electronic signals that may be transmitted to the device being remote from the mobile device and used for carrying out the method according to the disclosure. Typically, said means for data acquisition comprise at least one sensor. It will be understood that more than one sensor can be used in the mobile device, i.e., at least two, at least three, at least four, at least five, at least six, at least seven, at least eight, at least nine or at least ten or even more different sensors. Typical sensors used as means for data acquisition are sensors such as gyroscope, magnetometer, accelerometer, proximity sensors, thermometer, pedometer, fingerprint detectors, touch sensors, voice recorders, light sensors, pressure sensors, location data detectors, cameras, GPS, and the like. The evaluation unit typically comprises a processor and a database as well as software which is tangibly embedded to said device and, when running on said device, carries out the method of the disclosure. More typically, such a mobile device may also comprise a user interface, such as a screen, which allows for providing the result of the analysis carried out by the evaluation unit to a user.

Alternatively, it may be carried out on a device being remote with respect to the mobile device that has been used to acquire the said dataset. In this case, the mobile device shall merely comprise means for data acquisition, i.e., means which detect or measure either quantitatively or qualitatively physical parameters and transform them into electronic signals transmitted to the device being remote from the mobile device and used for carrying out the method according to the disclosure. Typically, said means for data acquisition comprise at least one sensor. It will be understood that more than one sensor can be used in the mobile device, i.e., at least two, at least three, at least four, at least five, at least six, at least seven, at least eight, at least nine or at least ten or even more different sensors. Typical sensors used as means for data acquisition are sensors such as gyroscope, magnetometer, accelerometer, proximity sensors, thermometer, pedometer, fingerprint detectors, touch sensors, voice recorders, light sensors, pressure sensors, location data detectors, cameras, GPS, and the like. Thus, the mobile device and the device used for carrying out the method of the disclosure may be physically different devices. In this case, the mobile device may communicate with the device used for carrying out the method of the present disclosure by any means for data transmission. Such data transmission may be achieved by a permanent or temporary physical connection, such as coaxial, fiber, fiber-optic or twisted-pair, 10 BASE-T cables. Alternatively, it may be achieved by a temporary or permanent wireless connection using, e.g., radio waves, such as Wi-Fi, LTE, LTE-advanced or Bluetooth. Accordingly, for carrying out the method of the present disclosure, the only requirement is the presence of a dataset comprising usage data for a mobile device within a first predefined time window obtained from a subject using a mobile device. The said dataset may also be transmitted or stored from the acquiring mobile device on a permanent or temporary memory device which subsequently can be used to transfer the data to the device used for carrying out the method of the present disclosure. The remote device which carries out the method of the disclosure in this setup typically comprises a processor and a database as well as software which is tangibly embedded to said device and, when running on said device, carries out the disclosed method. More typically, the said device may also comprise a user interface, such as a screen, which allows for providing the result of the analysis carried out by the evaluation unit to a user.

The term “assessing” as used herein refers to determining or providing an aid for diagnosing whether a subject suffers from schizophrenia or autism spectrum disorders and/or exhibits one or more negative symptoms associated therewith. Typically, assessing as referred to herein comprises determining an improvement, persistency or worsening of said negative symptoms, more typically an improvement of the said negative symptoms. As will be understood by those skilled in the art, such an assessment, although preferred to be, may usually not be correct for 100% of the investigated subjects. The term, however, requires that a statistically significant portion of subjects can be correctly assessed. Whether a portion is statistically significant can be determined by the person skilled in the art using various well known statistical evaluation tools, e.g., determination of confidence intervals, p-value determination, Student's t-test, Mann-Whitney test, etc. Details may be found in Dowdy and Wearden, Statistics for Research, John Wiley & Sons, New York 1983. Typically envisaged confidence intervals are at least 50%, at least 60%, at least 70%, at least 80%, at least 90% or at least 95%. The p-values are, typically, 0.2, 0.1, 0.05. Thus, the method of the present disclosure, typically, aids the assessment of schizophrenia or autism spectrum disorders by providing a means for evaluating a dataset comprising usage data for a mobile device within a first predefined time window. The term also encompasses any kind of diagnosing, monitoring or staging of schizophrenia or autism spectrum disorders.

In an embodiment of the method of this disclosure, said assessing schizophrenia comprises assessing at least one negative symptom associated with schizophrenia selected from the group consisting of: asociality, alogia, apathy, anhedonia and impaired attention. Typically, said assessing schizophrenia comprises determining an improvement of the at least one negative symptom associated with schizophrenia.

In yet another embodiment of the method of this disclosure, said assessing autism spectrum disorders comprises assessing at least one symptom associated with autism spectrum disorders selected from the group consisting of: social communication and social interaction, and restricted, repetitive patterns of behavior, interests or activities. Typically, said assessing autism spectrum disorders comprises determining an improvement of the at least one symptom associated with autism spectrum disorders.

The term “schizophrenia” as used herein refers to a mental disorder which is characterized by an abnormal behavior and an impaired ability to understand reality. Typical negative symptoms include asociality, alogia, apathy, anhedonia and impaired attention. Subjects suffering from schizophrenia may also have additional mental problems such as anxiety, depression or drug-abuse disorders. The symptoms typically are first apparent in young adulthood, and gradually worsen over a long time. Several causes for schizophrenia have been discussed including environmental causes, such as drug-abuse, nutrition during pregnancy or infections, or genetic causes. The term as used herein encompasses all subtypes of schizophrenia, i.e., the paranoid type, disorganized type, catatonic type, undifferentiated type, residual type, post-schizophrenic depression type, simple-schizophrenia type or other-schizophrenia type (see DSM 5 or ICD-10).

Schizophrenia may be, typically, diagnosed by interviewing the subject by a mental health specialist, applying the positive and negative syndrome scale (PANSS) or the brief negative symptom scale (BNSS).

Therapeutic measures for schizophrenia include treatment with antipsychotic drugs, such as aripiprazole, asenapine, brexpiprazole, cariprazine, chlorpromazine, fluphenazine, iloperidone, loxapine, lurasidone, molindone, paliperidone, perphenazine, prochlorperazine, risperidone, trifluoperazine, amisulpride, olanzapine, quetiapine, haloperidole, and clozapine, or physical therapies. Moreover, psychological and/or social counselling are also suitable measures.

The term “autism spectrum disorder” as used herein refers to a group of neurodevelopmental disorders including autism and related medical conditions. Typical symptoms include problems in social communication and social interaction, and restricted, repetitive patterns of behavior, interests or activities. Symptoms are usually recognized between one and two years of age. Long term issues may include difficulties in creating and keeping relationships, maintaining a job, and performing daily tasks. The DSM 5 recognizes autism, Asperger syndrome, pervasive developmental disorder not otherwise specified (PDD-NOS), and childhood disintegrative disorder as disorders falling into the group of autism spectrum disorders. Genetic reasons as well as environmental influences are discussed as potential risk factors.

Drugs which may be used for treating autism spectrum disorder patients include neurotransmitter reuptake inhibitors (fluoxetine), tricyclic antidepressants (imipramine), anticonvulsants (lamotrigine), atypical antipsychotics (clozapine), and acetylcholinesterase inhibitors (rivastigmine).

The term “subject” as used herein, typically, relates to mammals. The subject in accordance with the present disclosure may, typically, suffer from or shall be suspected to suffer from schizophrenia or autism spectrum disorders, i.e., it may already show some or all of the negative symptoms associated with the said diseases.

In an embodiment of the method of the disclosure said subject is a human.

The term “at least one usage behavior parameter” means that one or more usage behavior parameters may be determined in accordance with the disclosure, i.e., at least two, at least three, at least four, at least five, at least six, at least seven, at least eight, at least nine or at least ten or even more different performance parameters. Thus, there is no upper limit for the number of different usage behavior parameters which can be determined in accordance with the method of the present disclosure. Typically, however, there will be between one and twelve different usage behavior parameters determined per dataset of mobile device usage data.

The term “usage behavior parameter” as used herein refers to a parameter which is indicative of the usage behavior of a subject with respect to the mobile device. This typically includes the behavior of the subject more generally that is measured when the subject is wearing or carrying the device. For example, the mobile device in accordance with the present disclosure may be a smartphone. The dataset to be applied in accordance with the present disclosure shall comprise usage data for said smartphone recorded over a predefined period of time. Based on said data, usage behavior parameters may be calculated which reflect the usage behavior of the subject with respect to the smartphone, e.g., the frequency, kind of usage or non-usage (passive usage) or usage intensity etc. More typically, the usage behavior parameter(s) shall be recorded variables selected in the case schizophrenia from Table 1 and/or Table 2, below, or in the case of an autism spectrum disorder, from Table 4 and/or 5, below, i.e., are selected from the group consisting of: phone and app usage parameters, in particular, Contacts (number of IDs), Calls (frequency, time, duration, direction (i.e., incoming or outgoing calls)), Messages SMS (frequency, number of characters used, duration, direction), application (App) usage (name of the App, frequency, time, duration), Screen in use (frequency, time, duration), WIFI and/or bluetooth use (number of visible WIFI and/or bluetooth connections, number of used connections), ambient sound parameters, in particular, volume and pitch (volume power, time), speech classifier (frequency, time, duration), Mel-frequency cepstral coefficients, movement parameters, in particular, activity parameters (tri-axial acceleration (20 Hz), time), location (obfuscated GPS, i.e., distance and direction of travelling), and light and proximity parameters (amount of ambient light over time, proximity of objects over time). Moreover, the touch behavior parameters may be used as behavior parameter(s) in accordance with the method of the present disclosure. Typically, touch interactions, in particular, touch down, swiping and touch up, length and directionality of the touch movement, Y-coordinate of the touch event only, time stamps, whether or not it occurred on the keyboard, and/or typing behavior, in particular, character type (letter, number, punctuation mark, editing characters, function key, emoji), actual character used only for the following character types: punctuation mark (e.g., full stops, exclamation marks, editing characters (e.g., space, delete, backspace), time stamps may be envisaged. More typically, the usage behavior parameter(s) shall, thus, be recorded variables selected from Table 3, below, in the case of schizophrenia or Table 6, below in the case of an autism spectrum disorder.

More typically, the at least one usage behavior parameter may be a combination of the aforementioned parameters. The following combinations may, e.g., be envisaged:

phone and app usage parameters, ambient sound, movement parameters, and light and proximity parameters;

phone and app usage parameters, movement parameters, and light and proximity parameters;

phone and app usage parameters, ambient sound, and light and proximity parameters;

phone and app usage parameters, ambient sound, and movement parameters;

ambient sound, movement parameters, and light and proximity parameters;

phone and app usage parameters and ambient sound;

phone and app usage parameters, and movement parameters;

phone and app usage parameters, and light and proximity parameters;

ambient sound, and movement parameters;

ambient sound, and light and proximity parameters.

In an embodiment, the at least one behavior parameter is any of the aforementioned combinations in combination with a touch behavior parameter as set forth above.

In an embodiment of the method of the disclosure, said at least one usage behavior parameter is a recorded variable according to Table 1, 2 and/or 3, below, in the case of schizophrenia or Table 4, 5 and/or 6 in the case of an autism spectrum disorder.

The term “dataset comprising usage data for a mobile device” refers to an entirety of data reflecting or indicating different uses or tasks carried out by or with the mobile device which have been recorded by or acquired from the mobile device within a first time window. The first time window as referred to in this context is a predefined time window wherein the subject uses or is suspected to use the mobile device, i.e., it is the time period during which the dataset is recorded or acquired. Usage data may be, typically, phone usage data, application (App) usage data, ambient noise data, movement capture data and/or location capture data. The first time window may be of any length which is suitable for recording data that can be used for deriving a meaningful at least one usage behavior parameter. For example, if the duration of a phone call shall be measured, the said first time window shall at least last over said phone call. Typically, the usage data are recorded over a standardized time window, e.g., one or more hour(s), one or more day(s), one or more week(s) or one or more month(s). Depending on the subject and the circumstances, the skilled artisan is well aware of how to select a suitable redefined first time window for the purpose of recording or acquiring a dataset comprising mobile device usage data.

In an embodiment of the method of the disclosure, the said usage data for a mobile device may include data selected from the group consisting of: phone usage data, application (App) usage data, ambient noise data, movement capture data and location capture data.

The term “mobile device” as used herein refers to any portable device which comprises at least one sensor and data-recording equipment suitable for obtaining the dataset comprising usage data for a mobile device. This may also require a data processor and storage unit, voice recording devices, speakers, as well as a display for receiving input from the subject on the mobile device. Moreover, from the activity of the subject, data shall be recorded and compiled to a dataset which is to be evaluated by the method of the present disclosure either on the mobile device itself or on a second device. Depending on the specific setup envisaged, it may be necessary that the mobile device comprises data transmission equipment in order to transfer the acquired dataset from the mobile device to further device. Smartphones, portable multimedia devices or tablet computers are particularly well-suited as mobile devices according to the present disclosure. Alternatively, portable sensors with data recording and processing equipment may be used.

In an embodiment of the method of the disclosure, said mobile device is a smartphone, smartwatch, wearable sensor, portable multimedia device or tablet computer.

Determining at least one usage behavior parameter can be achieved either by directly deriving a desired measured value from the comprising usage data for a mobile device within a first predefined time window wherein said mobile device has been used by the subject. Alternatively, the usage behavior parameter may integrate one or more measured values from the dataset and, thus, may be derived from the dataset by mathematical operations such as calculations. Typically, the performance parameter is derived from the dataset by an automated algorithm, e.g., by a computer program which automatically derives the usage behavior parameter from the dataset when tangibly embedded on a data processing device fed by the said dataset.

The term “reference” as used herein refers to a discriminator which allows assessing schizophrenia or an autism spectrum disorder and, preferably, an improvement of the negative symptoms associated therewith in a subject. Such a discriminator may be a value for the usage behavior parameter which is indicative for subjects suffering from schizophrenia or an autism spectrum disorder and, preferably, exhibiting the negative symptoms associated therewith or not suffering from schizophrenia or an autism spectrum disorder and, preferably, the negative symptoms associated therewith.

In principle, such a value for a reference may be derived from a subject or group of subjects known to suffer from schizophrenia or an autism spectrum disorder and, in particular, exhibiting the negative symptoms associated therewith. If the determined usage behavior parameter is identical to the reference or above a threshold derived from the reference, the subject can be identified as suffering from schizophrenia or an autism spectrum disorder and, preferably, the negative symptoms associated therewith. If the determined usage behavior parameter differs from the reference and, in particular, is below the said threshold, the subject shall be identified as not suffering from or having an improvement of schizophrenia or an autism spectrum disorder or at least having an improvement of the negative symptoms associated therewith.

Alternatively, it may be derived from a subject or group of subjects known not to suffer from schizophrenia or an autism spectrum disorder and, in particular, not exhibiting the negative symptoms associated therewith. If the determined performance parameter from the subject is identical to the reference or below a threshold derived from the reference, the subject can be identified as not suffering from or having an improvement of schizophrenia or an autism spectrum disorder or at least having an improvement of the negative symptoms associated therewith. If the determined performance parameter differs from the reference and, in particular, is above the said threshold, the subject shall be identified as suffering from schizophrenia or an autism spectrum disorder and, preferably, the negative symptoms associated therewith.

More typically, the reference may be a previously determined usage behavior parameter from a comprising usage data for a mobile device within a second predefined time window wherein said mobile device has been used by the subject, wherein said second time window has been prior to the first time window. In such a case, a determined usage behavior parameter from the actual dataset which differs with respect to the previously determined usage behavior parameter shall be indicative for either an improvement or worsening depending on the previous status of the disease or a symptom accompanying it and the kind of usage represented by the usage behavior parameter. The skilled person knows based on the kind of usage and previous usage behavior parameter how the said parameter can be used as a reference. Typical differences between determined usage behavior parameters and references are reflected by the expected changes for the recorded variables being indicative for an improvement listed in Table 1, or 2 and/or 3, below, in the case of schizophrenia or Table 4, 5 and/or 6 in the case of an autism spectrum disorder.

Typically, an improvement of at least one negative symptom associated with schizophrenia or an autism spectrum disorder is determined if the at least one usage behavior parameter improves compared to the reference as indicated in Table 1, or 2 and/or 3, below, in the case of schizophrenia or Table 4, 5 and/or 6 in the case of an autism spectrum disorder.

In an embodiment of the method of the disclosure said reference is at least one usage behavior parameter which has been determined in a dataset comprising usage data for a mobile device within a second predefined time window prior to the first predefined time widow. The first and second time windows may be separated by a third predefined time period, i.e., a predefined monitoring period. Typically, such a period may also, depending on the length of the first and second time windows, range from days to weeks to months to years depending on the disease progression, state or development or the duration of therapeutic measures for the individual subject.

Comparing the determined at least one usage behavior parameter to a reference can be achieved by an automated comparison algorithm implemented on a data processing device such as a computer. The values of a determined usage behavior parameter and a reference for said determined usage behavior parameter, as specified elsewhere herein in detail, are compared to each other. As a result of the comparison, it can be assessed whether the determined usage behavior parameter is identical or differs from or is in a certain relation to the reference (e.g., is larger or lower than the reference). Based on said assessment, the subject can be identified as suffering from schizophrenia and, preferably, exhibiting the negative symptoms associated therewith (“rule-in”), or not (“rule-out”). For the assessment, the kind of reference will be taken into account as described elsewhere in connection with suitable references according to the disclosure.

Moreover, it will be understood by the skilled artisan that the aforementioned embodiments as well as those specified herein below are meant to refer to schizophrenia if schizophrenia shall be assessed whereas they are meant to refer to an autism spectrum disorder if the said autism spectrum disorder shall be assessed.

Moreover, by determining the degree of difference between a determined usage behavior parameter and a reference, a quantitative assessment of schizophrenia or an autism spectrum disorder shall be possible. It is to be understood that an improvement, worsening or unchanged overall disease condition or of symptoms thereof can be determined by comparing an actually determined usage behavior parameter to an earlier determined one used as a reference. Based on quantitative differences in the value of the said usage behavior parameter the improvement, worsening or unchanged condition can be determined and, optionally, also quantified. If other references, such as references from subjects with schizophrenia or an autism spectrum disorder are used, it will be understood that the quantitative differences are meaningful if a certain disease stage can be allocated to the reference collective. Relative to this disease stage, worsening, improvement or unchanged disease condition can be determined in such a case and, optionally, also quantified.

The assessment of schizophrenia or an autism spectrum disorder in the subject, is indicated to the subject or another person, such as a medical practitioner. Typically, this is achieved by displaying the assessment result on a display of the mobile device or the evaluation device. Alternatively, a recommendation for a therapy, such as a drug treatment, or for a certain life style, e.g., a certain nutritional diet or rehabilitation measures, is provided automatically to the subject or other person. To this end, the established diagnosis is compared to recommendations allocated to different diagnosis in a database. Once the established diagnosis matches one of the stored and allocated diagnoses, a suitable recommendation can be identified due to the allocation of the recommendation to the stored diagnosis matching the established diagnosis. Accordingly, it is, typically, envisaged that the recommendations and diagnoses are present in the form of a relational database. However, other arrangements which allow for the identification of suitable recommendations are also possible and known to the skilled artisan.

Thus, the method of the present disclosure in an embodiment also encompasses determining whether a schizophrenia or an autism spectrum disorder therapy or a therapy for at least the negative symptoms associated therewith was successful, or not.

In such a case, typically, between the second and the first time window the subject has received a schizophrenia or an autism spectrum disorder therapy or a therapy for at least the negative symptoms associated therewith. More typically, said therapy is a drug-based therapy.

An improvement of at least one negative symptom associated with schizophrenia or an autism spectrum disorder is, typically, indicative for a successful therapy.

Moreover, the one or more usage behavior parameter may also be stored on the mobile device or indicated to the subject, typically, in real-time. The stored usage behavior parameter may be assembled into a time course or similar evaluation measures. Such evaluated performance parameters may be provided to the subject as a feedback for usage behavior investigated in accordance with the method of the disclosure. Typically, such a feedback can be provided in electronic format on a suitable display of the mobile device and can be linked to a recommendation for a therapy as specified above or rehabilitation measures.

Further, the evaluated usage behavior parameter may also be provided to medical practitioners in doctors' offices or hospitals as well as to other health care providers, such as developers of diagnostic tests or drug developers in the context of clinical trials, health insurance providers or other stakeholders of the public or private health care system.

Typically, the method of the present disclosure for assessing schizophrenia or an autism spectrum disorder in a subject may be carried out as follows:

First, a usage behavior parameter is determined from an existing dataset comprising usage data for a mobile device within a first predefined time window wherein said mobile device has been used by the subject. Said dataset may have been transmitted from the mobile device to an evaluating device, such as a computer, or may be processed in the mobile device in order to derive the usage behavior parameter from the dataset.

Second, the determined usage behavior parameter is compared to a reference by, e.g., using a computer-implemented comparison algorithm carried out by the data processor of the mobile device or by the evaluating device, e.g., the computer. The result of the comparison is assessed with respect to the reference used in the comparison and based on the said assessment the subject will be identified as a subject suffering schizophrenia or an autism spectrum disorder, or not, or exhibiting an improvement of the negative symptoms associated therewith, or not.

Third, the said result of the assessment is indicated to the subject or to another person, such as a medical practitioner. However, it will be understood that for a final clinical diagnosis or assessment further factors or parameters may be taken into account by the clinician.

Further, a recommendation for a therapy is provided automatically to the subject or another person. To this end, the established diagnosis is compared to recommendations allocated to different diagnosis in a database. Once the established diagnosis matches one of the stored and allocated diagnoses, a suitable recommendation can be identified due to the allocation of the recommendation to the stored diagnosis matching the established diagnosis. Typical recommendations in the case of schizophrenia involve therapy with antipsychotic drugs, such as aripiprazole, asenapine, brexpiprazole, cariprazine, chlorpromazine, fluphenazine, iloperidone, loxapine, lurasidone, molindone, paliperidone, perphenazine, prochlorperazine, risperidone, trifluoperazine, amisulpride, olanzapine, quetiapine, haloperidole, and clozapine, or physical therapies or in the case of an autism spectrum disorder neurotransmitter reuptake inhibitors (fluoxetine), tricyclic antidepressants (imipramine), anticonvulsants (lamotrigine), atypical antipsychotics (clozapine), and acetylcholinesterase inhibitors (rivastigmine). Moreover, psychological and/or social counselling are also suitable measures.

As an alternative or in addition, the usage behavior parameter underlying the diagnosis will be stored on the mobile device. Typically, it shall be evaluated together with other stored performance parameters by suitable evaluation tools, such as time course assembling algorithms, implemented on the mobile device which can assist electronically with therapy recommendations as specified elsewhere herein.

The disclosure, in light of the above, also specifically contemplates a method of assessing schizophrenia or an autism spectrum disorder and, preferably, an improvement of the negative symptoms associated therewith in a subject comprising the steps of:
a) obtaining from said subject using a mobile device a dataset comprising usage data for a mobile device within a first predefined time window wherein said mobile device has been used by the subject;
b) determining at least one usage behavior parameter determined from said dataset;
c) comparing the determined at least one usage behavior parameter to a reference; and
d) assessing schizophrenia or an autism spectrum disorder and, preferably, an improvement of the negative symptoms associated therewith in a subject based on the comparison carried out in step (b), typically, by determining whether the subject suffers from schizophrenia or an autism spectrum disorder or exhibits an improvement of the negative symptoms associated therewith, or not.

Advantageously, it has been found in the studies underlying the present disclosure that usage behavior parameters obtained from datasets comprising usage data for a mobile device within a first predefined time window wherein said mobile device has been used by the subject can be used to assess schizophrenia or an autism spectrum disorder in said subject. In particular, the said usage behavior parameters can be used to identify an improvement of the negative symptoms associated with schizophrenia or an autism spectrum disorder in said subject and, thus, aid monitoring of subjects, e.g., undergoing a schizophrenia or an autism spectrum disorder therapy as specified elsewhere herein. The said datasets can be acquired from schizophrenia or an autism spectrum disorder patients in a convenient manner by using mobile devices such as the omnipresent smart phones, portable multimedia devices or tablet computers on which the subjects perform active or passive pressure tests. The datasets acquired can be subsequently evaluated by the method of the disclosure for the usage behavior parameter suitable as digital biomarker. Said evaluation can be carried out on the same mobile device or it can be carried out on a separate remote device. Moreover, by using such mobile devices, recommendations on therapeutic measures can be provided to the patients directly, i.e., without the consultation of a medical practitioner in a doctor's office or hospital or emergency medical provider. Thanks to the present disclosure, the life conditions of schizophrenia or autism spectrum disorder patients can be adjusted more precisely to the actual disease status due to the use of actual determined usage behavior parameter by the method of the disclosure. Thereby, drug treatments can be evaluated for efficacy and dosage regimens can be adapted to the current status of the patient. It is to be understood that the method of this disclosure is, typically, a data evaluation method which requires an existing dataset from a subject. Within this dataset, the method determines at least one usage behavior parameter which can be used for assessing schizophrenia or an autism spectrum disorder.

Accordingly, the method of the present disclosure may be used for:
assessing the disease condition;
monitoring patients, in particular, in a real life, daily situation and on a large scale;
supporting patients with therapy recommendations;
investigating drug efficacy, e.g., also during clinical trials;
facilitating and/or aiding therapeutic decision making;
supporting hospital management;
supporting health insurance assessments and management; and/or
supporting decisions in public health management.

The present disclosure also contemplates a computer program, computer program product or computer readable storage medium having tangibly embedded thereon said computer program, wherein the computer program comprises instructions that, when run on a data processing device or computer, carry out the method of the present disclosure as specified above. Specifically, the present disclosure further encompasses:
A computer or computer network comprising at least one processor, wherein the processor is adapted to perform the method according to one of the embodiments of this disclosure,
a computer loadable data structure that is adapted to perform the method according to one of the embodiments described herein while the data structure is being executed on a computer,
a computer script, wherein the computer program is adapted to perform the method according to one of the embodiments described herein while the program is being executed on a computer,
a computer program comprising program means for performing the method according to one of the embodiments described herein while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this disclosure after having been loaded into a main and/or working storage of a computer or of a computer network,
a computer program product having program code means, wherein the program code means can be stored or are stored on a storage medium, for performing the method according to one of the embodiments described herein, if the program code means are executed on a computer or on a computer network,
a data stream signal, typically encrypted, comprising a dataset comprising usage data for a mobile device within a first predefined time window wherein said mobile device has been used by the subject, and
a data stream signal, typically encrypted, comprising the at least one usage behavior parameter derived from the dataset.

The present disclosure, further, relates to a method for determining at least one usage behavior parameter from a dataset comprising usage data for a mobile device within a first predefined time window wherein said mobile device has been used by the subject:
a) deriving at least one usage behavior parameter from said dataset; and
b) comparing the determined at least one usage behavior parameter to a reference, wherein, typically, said at least one usage behavior parameter can aid assessing schizophrenia or an autism spectrum disorder and, preferably, assessing an improvement of the negative symptoms associated therewith in said subject.

In an embodiment, the present disclosure, moreover, contemplates a method for the treatment of schizophrenia using the usage behavior parameters specified herein in combination with known therapeutic measures for the treatment of schizophrenia. In particular, this relates to a method for the treatment of schizophrenia comprising the steps of:
a) determining at least one usage behavior parameter from a dataset comprising usage data for a mobile device within a first predefined time window wherein said mobile device has been used by the subject;
b) comparing the determined at least one usage behavior parameter to a reference, whereby schizophrenia will be assessed; and
c) administering therapeutic measures for schizophrenia to the subject.

Preferably, therapeutic measures for schizophrenia include treatment with antipsychotic drugs, such as aripiprazole, asenapine, brexpiprazole, cariprazine, chlorpromazine, fluphenazine, iloperidone, loxapine, lurasidone, molindone, paliperidone, perphenazine, prochlorperazine, risperidone, trifluoperazine, amisulpride, olanzapine, quetiapine, haloperidole, and clozapine, or physical therapies. Moreover, psychological and/or social counselling are also suitable measures.

In a further embodiment, the present disclosure contemplates usage behavior parameters in combination with the above therapeutic measures for schizophrenia for use in the treatment of schizophrenia.

The present disclosure relates to a mobile device comprising a processor, at least one sensor recording usage data and a database as well as software which is tangibly embedded to said device and, when running on said device, carries out the method of the disclosure.

The said mobile device is, thus, configured to be capable of acquiring the dataset and to determine the usage behavior parameter therefrom. Moreover, it is configured to carry out the comparison to a reference and to establish the assessment of schizophrenia or an autism spectrum disorder as described elsewhere herein in detail.

In a further embodiment, the present disclosure contemplates said mobile device in combination with the above therapeutic measures for schizophrenia for use in the treatment of schizophrenia.

In a still further embodiment, the present disclosure relates to antipsychotic drugs for schizophrenia for use in a method for the treatment of schizophrenia, wherein the efficacy of the antipsychotic drug is investigated and/or adapted using the method assessing schizophrenia as specified herein.

In a still further embodiment, the present disclosure relates to antipsychotic drugs for schizophrenia for use in a method for the treatment of schizophrenia, wherein the patient is monitored using the method assessing schizophrenia as specified herein.

Antipsychotic drugs for schizophrenia may preferably include aripiprazole, asenapine, brexpiprazole, cariprazine, chlorpromazine, fluphenazine, iloperidone, loxapine, lurasidone, molindone, paliperidone, perphenazine, prochlorperazine, risperidone, trifluoperazine, amisulpride, olanzapine, quetiapine, haloperidole, and clozapine.

The present disclosure further relates to a system comprising a mobile device comprising at least one sensor recording usage data and a remote device comprising a processor and a database as well as software which is tangibly embedded on said device and, when running on said device, carries out the method of the disclosure, wherein said mobile device and said remote device are operatively linked to each other.

Under “operatively linked to each other” it is to be understood that the devices are connected as to allow data transfer from one device to the other device. Typically, it is envisaged that at least the mobile device which acquires data from the subject is connected to the remote device carrying out the steps of the methods of the disclosure such that the acquired data can be transmitted to the remote device for processing. However, the remote device may also transmit data to the mobile device such as signals controlling or supervising its proper function. The connection between the mobile device and the remote device may be achieved by a permanent or temporary physical connection, such as coaxial, fiber, fiber-optic or twisted-pair, 10 BASE-T cables. Alternatively, it may be achieved by a temporary or permanent wireless connection using, e.g., radio waves, such as Wi-Fi, LTE, LTE-advanced or Bluetooth. Further details may be found elsewhere in this specification. For data acquisition, the mobile device may comprise a user interface such as screen or other equipment for data acquisition.

The present disclosure further contemplates the use of the mobile device or the system of the disclosure for assessing schizophrenia or an autism spectrum disorder analyzing a dataset comprising usage data for a mobile device within a first predefined time window wherein said mobile device has been used by the subject.

The present disclosure also contemplates a method assessing the neurological status of patients with psychiatric, neurodevelopmental, neurodegenerative, neuromuscular and neurological auto-immune disorders:
a) determining at least one usage behavior parameter from a dataset comprising usage data for a mobile device within a first predefined time window wherein said mobile device has been used by the subject; and
b) comparing the determined at least one usage behavior parameter to a reference, whereby the neurological status of patients with psychiatric, neurodevelopmental, neurodegenerative, neuromuscular and neurological auto-immune disorders will be assessed.

Further contemplated is a method assessing the neurological status of patients with schizophrenia, bipolar disorder, depression, autism spectrum disorder, Parkinson's disease, Alzheimer's disease, Huntington's disease, spinal muscular atrophy, amyotrophic lateral sclerosis, Duchene muscular dystrophy, multiple sclerosis:
a) determining at least one usage behavior parameter from a dataset comprising usage data for a mobile device within a first predefined time window wherein said mobile device has been used by the subject; and
b) comparing the determined at least one usage behavior parameter to a reference, whereby the neurological status of patients with schizophrenia, bipolar disorder, depression, autism spectrum disorder, Parkinson's disease, Alzheimer's disease, Huntington's disease, spinal muscular atrophy, amyotrophic lateral sclerosis, Duchene muscular dystrophy, multiple sclerosis will be assessed.

In the following, diseases are mentioned which could also be assessed by the method of the present disclosure:

Parkinson's disease comprises assessing at least one symptom associated with Parkinson's disease selected from a group consisting of: bradykinesia, tremor, rigidity, dyskinesias, involuntary movements, speech difficulties, gait problems and walking difficulty, fatigue and changes to diurnal rhythms, cognitive impairment of processing speed, attention.

Huntington's disease comprises assessing at least one symptom associated with Huntington's disease selected from a group consisting of: Psychomotor slowing, chorea (jerking, writhing), progressive dysarthria, rigidity and dystonia, social withdrawal, progressive cognitive impairment of processing speed, attention, planning, visual-spatial processing, learning (though intact recall), fatigue and changes to diurnal rhythms.

Spinal muscular atrophy, comprises assessing at least one symptom associated with Spinal muscular atrophy selected from a group consisting of: hypotonia and muscle weakness, fatigue and changes to diurnal rhythms.

Duchene muscular dystrophy, comprises assessing at least one symptom associated with Spinal muscular atrophy selected from a group consisting of: hypotonia and muscle weakness, gait abnormalities, cognitive developmental retardation of attention, verbal learning, memory and social communication and interaction, fatigue and changes to diurnal rhythms.

Amyotorphic lateral sclerosis, comprises assessing at least one symptom associated with Amyotorphic lateral sclerosis selected from a group consisting of: hypotonia and muscle weakness, problems with coordination, stiff muscles, loss of muscle, muscle spasms, or overactive reflexes.

Multiple sclerosis, comprises assessing at least one symptom associated with multiple sclerosis selected from a group consisting of: impaired fine motor abilities, pins and needs, numbness in the fingers, fatigue and changes to diurnal rhythms, gait problems and walking difficulty, cognitive impairment including problems with processing speed.

In the following, further particular embodiments are listed:

Embodiment 1. A method assessing schizophrenia or an autism spectrum disorder in a subject comprising the steps of:
a) determining at least one usage behavior parameter from a dataset comprising usage data for a mobile device within a first predefined time window wherein said mobile device has been used by the subject; and
b) comparing the determined at least one usage behavior parameter to a reference, whereby schizophrenia or an autism spectrum disorder will be assessed.

Embodiment 2. The method of embodiment 1, wherein said assessing schizophrenia comprises assessing at least one negative symptom associated with schizophrenia selected from the group consisting of: asociality, alogia, apathy, anhedonia and impaired attention and wherein said assessing an autism spectrum disorder comprises assessing at least one negative symptom associated with an autism spectrum disorder selected from the group consisting of: social communication and social interaction, and restricted, repetitive patterns of behavior, interests or activities.

Embodiment 3. The method of embodiment 2, wherein said assessing schizophrenia or an autism spectrum disorder comprises determining an improvement of the at least one negative symptom associated with schizophrenia or an autism spectrum disorder.

Embodiment 4. The method of any one of embodiments 1 to 3, wherein the said usage data for a mobile device comprise data selected from the group consisting of: phone usage data, application (App) usage data, ambient noise data, movement capture data and location capture data.

Embodiment 5. The method of any one of embodiments 1 to 4, wherein said at least one usage behavior parameter is a recorded variable according to Table 1, 2 and/or 3 in the case of schizophrenia, and Table 4, 5 and/or 6 in the case of an autism spectrum disorder.

Embodiment 6. The method of embodiment 5, wherein an improvement of at least one negative symptom associated with schizophrenia or an autism spectrum disorder is determined if the at least one usage behavior parameter improves compared to the reference as indicated in Table 1 2, and/or 3 in the case of schizophrenia, and Table 4, 5 and/or 6 in the case of an autism spectrum disorder.

Embodiment 7. The method of any one of embodiments 1 to 6, wherein said reference is at least one usage behavior parameter which has been determined in a dataset comprising usage data for a mobile device within a second predefined time window prior to the first predefined time widow.

Embodiment 8. The method of embodiment 7, wherein between the second and the first time window the subject has received a schizophrenia or an autism spectrum disorder therapy or a therapy for at least one of the negative symptoms associated therewith.

Embodiment 9. The method of embodiment 8, wherein said therapy is a drug-based therapy.

Embodiment 10. The method of embodiment 8 or 9, wherein an improvement of at least one negative symptom associated with schizophrenia or an autism spectrum disorder is indicative for a successful therapy.

Embodiment 11. The method of any one of embodiments 1 to 10, wherein said mobile device is a smartphone, smartwatch, wearable sensor, portable multimedia device or tablet computer.

Embodiment 12. The method of any one of embodiments 1 to 11, wherein said subject is a human.

Embodiment 13. A mobile device comprising a processor, at least one sensor recording usage data and a database as well as software which is tangibly embedded to said device and, when running on said device, carries out the method of any one of embodiments 1 to 12.

Embodiment 14. A system comprising a mobile device comprising at least one sensor recording usage data and a remote device comprising a processor and a database as well as software which is tangibly embedded to said device and, when running on said device, carries out the method of any one of embodiments 1 to 12, wherein said mobile device and said remote device are operatively linked to each other.

Embodiment 15. Use of the mobile device according to embodiment 13 or the system of embodiment 14 for assessing schizophrenia or an autism spectrum disorder analyzing a dataset comprising usage data for a mobile device within a first predefined time window wherein said mobile device has been used by the subject.

Embodiment 16. A method for the treatment of schizophrenia comprising the method of any one of embodiments 1 to 12, and further a step of:

c) administering therapeutic measures for schizophrenia to the subject.

Embodiment 17. Antipsychotic drugs for schizophrenia for use in a method for the treatment of schizophrenia, wherein the efficacy of the antipsychotic drug is investigated and/or adapted using the method of assessing schizophrenia of any one of embodiments 1 to 12.

All references cited throughout this specification are hereby incorporated herein by reference with respect to their entire disclosure content and with respect to the specific disclosure contents mentioned in the specification.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1A shows the activation of the App for data capture from patients informing the patient about the usage behavior that will be captured;

FIG. 1B shows the capture of contacts;

FIG. 1C shows phone calls and messages;

FIG. 1D shows App usage;

FIG. 1E shows ambient noise;

FIG. 1F shows location and movement;

FIG. 1G shows that the App will inform how data capture can be stopped or interrupted.

FIG. 2A shows a profile of captured phone usage data from a patient.

FIG. 2B shows a profile of captured app usage data from a patient.

FIG. 2C shows a profile of captured accelerometer data from a patient.

FIG. 2D shows a profile of captured ambient noise data from a patient.

### DESCRIPTION AND EXAMPLES

The embodiments and examples described below are not intended to be exhaustive or to limit the invention to the precise forms disclosed in the following detailed description. Rather, the embodiments are chosen and described so that others skilled in the art may appreciate and understand the principles and practices of this disclosure.

### EXAMPLES

### Example 1: Investigation of Mobile Phone Behavior Over 16 Weeks in Schizophrenia Patients

The smart phone usage behavior of 100 patients suffering from schizophrenia will be monitored over a period of 16 weeks (observation period). The patients will use Android-based smart phones. Patients may receive a drug. Smart phone usage which will be investigated includes phone usage, App usage, ambient noise, movement, location and general handling as well as touch behavior.

In the following Tables 1, 2 and/or 3, the captured data, the variables (usage parameters) and the expectations associated with an improvement of schizophrenia are indicated.

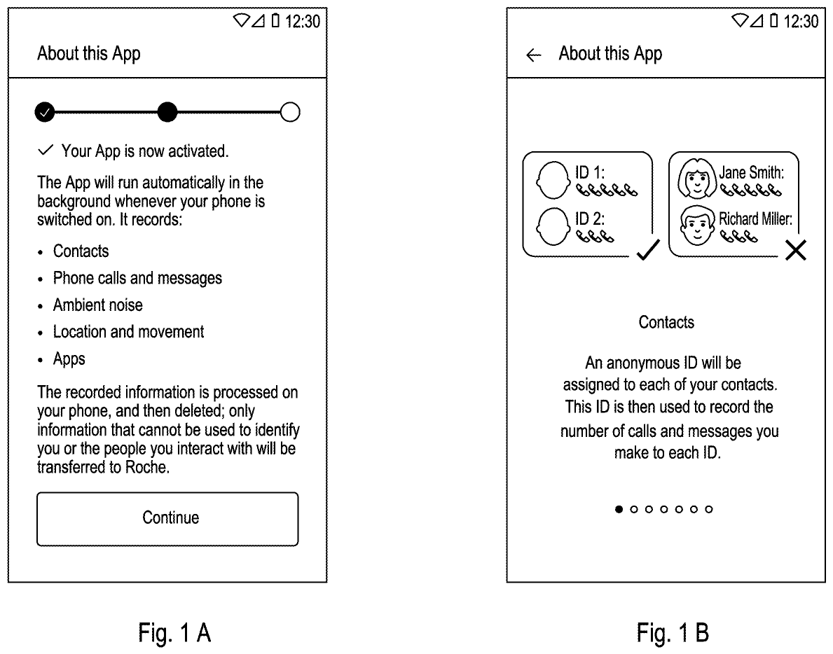

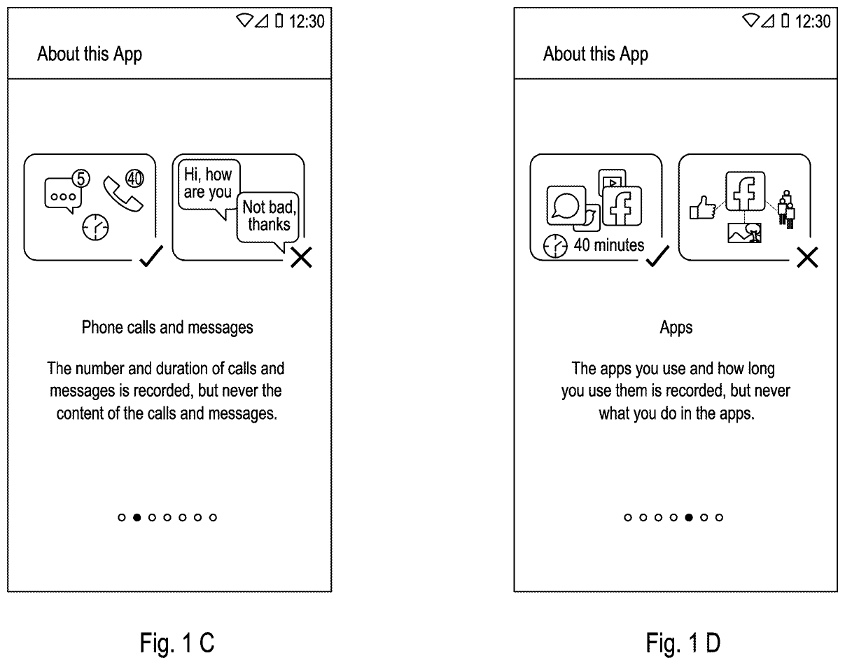

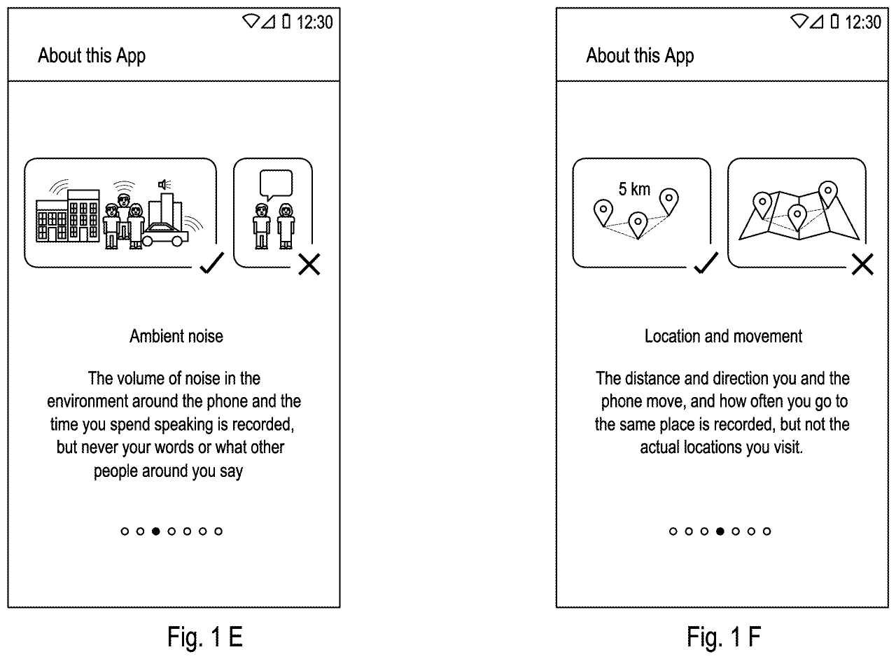

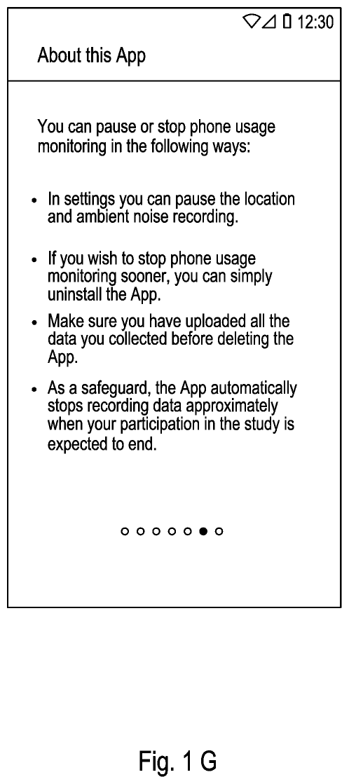

In order to capture the said usage data, an App will be installed on the smart phones of the patients. The App will automatically capture the usage behavior data within a certain time window, derive usage behavior parameters therefrom as indicated in Tables 1 and 2 and store these parameters on the smart phone. The data capture will be carried out several times during the observation period, e.g., each day. The App will inform the patient once data capture is started and when it ends (FIGS. 1A-1G). Moreover, in order to safeguard data protection provisions, the App will be activated by an investigator at the beginning of the observation period and de-installed by the said investigator at the end of the observation period. Only patients which have given their informed consent will be observed. All data which may be transferred before, during or after the observation period will be encrypted.

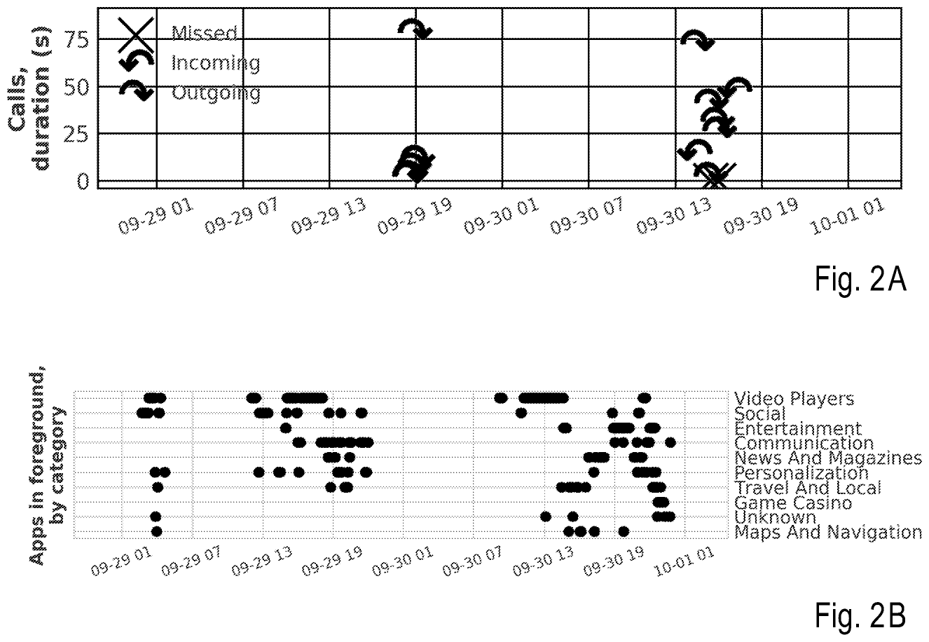

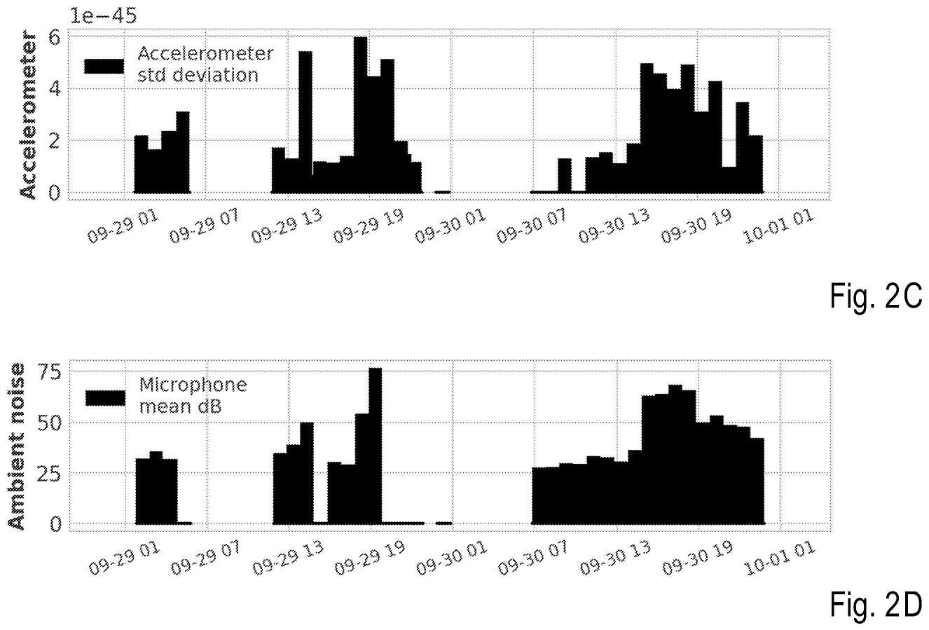

A profile of captured data from a patient is depicted in FIG. 2.

### Example 2: Investigation of Mobile Phone Behavior Over 16 Weeks in Autism Spectrum Disorder Patients

The smart phone usage behavior of 100 patients suffering from an autism spectrum disorder will be monitored over a period of 16 weeks (observation period). The patients will use Android-based smart phones. Patients may receive a drug. Smart phone usage which will be investigated includes phone usage, App usage, ambient noise, movement, location and general handling as well as touch behavior.

In the following Tables 4, 5 and/or 6, the captured data, the variables (usage parameters) and the expectations associated with an improvement of the autism spectrum disorder are indicated.

In order to capture the said usage data, an App will be installed on the smart phones of the patients. The App will automatically capture the usage behavior data within a certain time window, derive usage behavior parameters therefrom as indicated in Tables 4 and 5 and store these parameters on the smart phone. The data capture will be carried out several times during the observation period, e.g., each day. The App will inform the patient once data capture is started and when it ends (FIGS. 1A-1G). Moreover, in order to safeguard data protection provisions, the App will be activated by an investigator at the beginning of the observation period and de-installed by the said investigator at the end of the observation period. Only patients which have given their informed consent will be observed. All data which may be transferred before, during or after the observation period will be encrypted.

TABLE 1
Data for phone usage and ambient sound

| | | | Why we are recording this: |
|---|---|---|---|
| | | | We expect that patients with |
| | | | improvements in Schizophrenia Negative |
| Domain | Sub-domain | Variables being recorded | Symptoms (SNS) clinical scales will show |
| Phone and App Usage | Log | Each contact is assigned an | Increased the number of contacts they |
| | Contacts | anonymous ID. Calls and | call, phone call duration and number of |
| | | SMS are logged against this | characters |
| | | ID (see below) | |
| | Log Calls | Frequency, time, duration, | |
| | | incoming or outgoing | |
| | Log SMS | Frequency, time, duration, | |
| | | incoming or outgoing, | |
| | | number of characters | |
| | Log App | Name of App | Decreased the time and frequency of non- |
| | Usage | Frequency, time, duration of | social apps and/or games, while |
| | | App usage | increasing the frequency and time spend |
| | | | in Social apps. |
| | | | Overall, we expect the total amount of |
| | | | time spend using App will decrease. |
| | Log Screen | Frequency, time, duration | Decreased unlock duration every time the |
| | On | | patient use the phone |
| | Log WIFI & | Number of visible WIFI & | Increased number of networks (WIFI) |
| | bluetooth | Bluetooth | and devices (bluetooth) during the day |
| | | Number of WIFIs used | Decrease duration connected to the most |
| | | | used network (home) |
| | | | Increased duration connected to different |
| | | | networks |
| Ambient Sound | Audio is recorded for 10 seconds every minute, processed on the phone to compute | | |
| | the features below. | | |
| | The raw audio recordings are discarded once the features are computed. | | |
| | Volume & | Volume (power), time | Increased volume during the day, but |
| | pitch | | larger increases during the morning |
| | | | Higher pitch in voiced frames |
| | Speech | Frequency, time, duration | Increased ratio of voiced and non-voiced |
| | Classifier | | frames |
| | | | Increased duration in the voiced frames |
| | Sound | Mel-frequency Cepstral | (Required for further optimizing the speech |
| | power | Coefficients | classifier) |
| | spectrum | | |

TABLE 2
Data for movement and light & proximity

| | | | What we expect to show with these data: |
|---|---|---|---|
| | | | We expect that patients with |
| | | | improvements in Schizophrenia Negative |
| | | Variables | Symptoms (SNS) clinical scales will |
| Domain | Sub-domain | being recorded | show . . . |
| Movement | Activity | Tri-axial acceleration | Increased activity during the day |
| | Levels | (20 Hz), time | Decreased activity during the night |
| | | | Using motor behavior classification: |
| | | | Increased walk duration, longer walks |
| | | | Decreased duration of not moving |
| | | | Increased time on car travels |
| | Location | Obfuscated GPS, | Increased number of new places visited |
| | | i.e., distance and direction | More time spent in social places, |
| | | of travel | identified using ambient noise |
| | | | measures |
| | | | Longer distance covered during the day |
| | | | Reduced time spend in a single place |
| | | | (home) |
| Light & | Phone | Amount of ambient | Increased duration of the phone in the pocket |
| proximity | handling | light over time | Decreased duration of use of the phone |
| | classification | Proximity of objects | in the darkness |
| | | over time | |

TABLE 3
Data from touch behavior

| | | | What we expect to show with these |
|---|---|---|---|
| | | | data: |
| | | | We expect that patients with |
| | | | improvements in Schizophrenia |
| | | | Negative Symptoms (SNS) clinical |
| Domain | Sub-domain | Variables being recorded | scales will show . . . |
| Touch behavior | Touch | For every touch interaction: | Decreased amount of activity and |
| | interactions | Touch down, swiping and | interaction in non-social apps |
| | | touch up | and/or games, while increased |
| | | Length and directionality of the | interaction with social apps. |
| | | touch movement | Less browsing behavior in Apps, |
| | | Y-coordinate of the touch event | as measured by swipe gestures |
| | | only | Changes to the circadian rhythm, |
| | | Time stamps | i.e., less interactions at night/in |
| | | Whether it occurred on the | darkness |
| | | keyboard | |
| | Typing | For all characters entered on the | Increased amounts of typing |
| | behavior | screen via the keyboard: | behavior |
| | | Character type (letter, number, | Increased amounts of typing |
| | | punctuation mark, editing | behavior in social apps |
| | | characters, function key, emoji) | Increased used of certain |
| | | Actual character used only for | punctuation marks, e.g., question |
| | | the following character types: | marks and exclamation marks |
| | | punctuation mark (e.g., full stops, | Faster typing behavior |
| | | exclamation marks, editing | Changes to the circadian rhythm, |
| | | characters (e.g., space, delete, | i.e., less interactions at night/in |
| | | backspace) | darkness |
| | | Time stamps | |

TABLE 4
Data for phone usage and ambient sound

| | | | Why we are recording this: |
|---|---|---|---|
| | | | We expect that patients with improvements in |
| | | | Autism Spectrum Disorder Sociability and |
| | | | Communication domains of clinical scales that |
| | Sub- | | measure sociability (SRS-2, ADOS-2, |
| Domain | domain | Variables being recorded | VINELAND-II) will show . . . |
| Phone and | Anonymous ID generated for contacts, name, number and photo ID. | | |
| App Usage | This table is stored in device storage only. | | |
| | Log | Each contact is assigned an | Increased the number of contacts they call, phone |
| | Contacts | anonymous ID. Calls and | call duration and number of characters |
| | | SMS are logged against this | |
| | | ID (see below) | |
| | Log Calls | Frequency, time, duration, | |
| | | incoming or outgoing | |
| | Log SMS | Frequency, time, duration, | |
| | | incoming or outgoing, | |
| | | number of characters | |
| | Log App | Name of App | Decreased the time and frequency of non-social |
| | Usage | Frequency, time, duration of | apps and/or games, while increasing the frequency |
| | | App usage | and time spend in Social apps. |
| | | (foreground/background) | Overall, we expect the total amount of time spend |
| | | | using App will decrease. |
| | Log Screen | Frequency, time, duration | Decreased unlock duration every time the patient |
| | On | | use the phone |
| | Log WIFI | Number of visible WIFI & | Increased number of networks (WIFI) and devices |
| | & | Bluetooth | (bluetooth) during the day |
| | bluetooth | Number of WIFIs used | Decrease duration connected to the most used |
| | | | network (home) |
| | | | Increased duration connected to different |
| | | | networks |
| Ambient | Audio is recorded for 10 seconds every minute, processed on the phone to compute the | | |
| Sound | features below. Occurs in memory and is never stored. The raw audio recordings are | | |
| | discarded once the features are computed. | | |
| | Volume & | Volume (power), time | Increased volume during the day, but larger |
| | pitch | | increases during the morning |
| | | | Higher pitch in voiced frames |
| | Speech | Frequency, time, duration | Increased ratio of voiced and non-voiced frames |
| | Classifier | | Increased duration in the voiced frames |
| | Sound | Mel frequency Cepstral | (Required for further optimizing the speech |
| | power | Coefficients | classifier) |
| | spectrum | | |

TABLE 5
Data for movement and light & proximity

| | | | Why we are recording this: |
|---|---|---|---|
| | | | We expect that patients with |
| | | | improvements in Autism Spectrum |
| | | | Disorder Sociability and Communication |
| | | | domains of clinical scales that measure |
| | | | sociability (SRS-2, ADOS, VINELAND- |
| Domain | Sub-domain | Variables being recorded | II) will show . . . |
| Movement | Activity | Tri-axial acceleration | Increased activity during the day |
| | Levels | (20 Hz), time | Decreased activity during the night |
| | | | Using motor behavior classification: |
| | | | Increased walk duration, longer walks |
| | | | Decreased duration of not moving |
| | | | Increased time on car travels |
| | Location | Obfuscated GPS, i.e., | Increased number of new places visited |
| | | distance and direction of | More time spent in social places, |
| | | travel | identified using ambient noise measures |
| | | | Longer distance covered during the day |
| | | | Reduced time spend in a single place |
| | | | (home) |
| Light & | Phone | Amount of ambient light | Increased duration of the phone in the |
| proximity | handling | over time | pocket |
| | classification | Proximity of objects over | Decreased duration of use of the phone in |
| | | time | the darkness |
| Phone information | Technical | Android version of the | For technical diagnostics only |
| | phone | device | |
| | information | Battery Health | |
| | | Battery consumption | |
| | | Storage Space Total and | |
| | | consumed (intenal and SD | |
| | | card) | |
| | | Data size (study and non- | |
| | | study related) | |

TABLE 6
Data from touch behavior

| | | | Why we are recording this: |
|---|---|---|---|
| | | | We expect that patients with |
| | | | improvements in Autism Spectrum |
| | | | Disorder Sociability and Communication |
| | | | domains of clinical scales that measure |
| | | | sociability (SRS-2, ADOS, VINELAND- |
| Domain | Sub-domain | Variables being recorded | II) will show . . . |
| Touch | Touch | For every touch interaction: | Decreased amount of activity and |
| behavior | interactions | Touch down, swiping and | interaction in non-social apps and/or |
| | | touch up | games, while increased interaction with |
| | | Length and directionality | social apps. |
| | | of the touch movement | Less browsing behavior in Apps, as |
| | | Y-coordinate of the touch | measured by swipe gestures |
| | | event only | Changes to the circadian rhythm, i.e., less |
| | | Time stamps | interactions at night/in darkness |
| | | Whether it occurred on the | |
| | | keyboard | |
| | Typing | For all characters entered on | Increased amounts of typing behavior |
| | behavior | the screen via the keyboard: | Increased amounts of typing behavior in |
| | | Character type (letter, | social apps |
| | | number, punctuation mark, | Increased used of certain punctuation |
| | | editing characters, function | marks, e.g., question marks and |
| | | key, emoji) | exclamation marks |
| | | Actual character used only | Faster typing behavior |
| | | for the following character | Changes to the circadian rhythm, i.e., less |
| | | types: punctuation mark | interactions at night/in darkness |
| | | (e.g., full stops, exclamation | |
| | | marks, editing characters | |
| | | (e.g., space, delete, | |
| | | backspace) | |
| | | Time stamps | |

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

What is claimed is:

1. A method of assessing schizophrenia in a subject, comprising:
a) determining at least one usage behavior parameter from a dataset comprising usage data for a mobile device within a first predefined time window wherein said mobile device has been used by the subject;
b) comparing the determined at least one usage behavior parameter to a reference; and
c) assessing schizophrenia in the subject based on the comparison of step b).

2. The method of claim 1, wherein said assessing schizophrenia comprises assessing at least one negative symptom associated with schizophrenia selected from the group consisting of: asociality, alogia, apathy, anhedonia and impaired attention.

3. The method of claim 2, wherein said assessing schizophrenia comprises determining an improvement of the at least one negative symptom associated with schizophrenia.

4. The method of claim 1, wherein the usage data for a mobile device comprises data selected from the group consisting of: phone usage data, application (App) usage data, ambient noise data, movement capture data and location capture data.

5. The method of claim 1, wherein said at least one usage behavior parameter is a recorded variable selected from the group consisting of:
(i) logged app usage, logged screen on, and/or logged WiFi and bluetooth; and
(ii) touch behavior, touch interactions and/or typing behavior.

6. The method of claim 5, wherein an improvement of at least one negative symptom associated with schizophrenia is determined by improvements in the (i) logged app usage, logged screen on, logged WiFi and bluetooth, (ii) touch behavior, touch behavior, touch interactions and/or typing behavior:
(i) wherein the improvement in logged app usage, logged screen on, and/or logged WiFi and Bluetooth comprises decreased time and frequency of non-social apps and/or games, increased frequency and time spent in social apps, decrease of total amount of time spent using Apps; decreased unlock duration every time the patient uses the phone, increased number of networks (WIFI) and devices (bluetooth) during the day, decreased duration connected to the most used network (home), and/or increased duration connected to networks different from the most used network; and
(ii) wherein improvement in touch behavior, touch interactions and/or typing behavior comprises decreased activity and interaction in non-social apps and/or games; increased interaction with social apps; less browsing behavior in apps, as measured by swipe gestures; increased amounts of typing behavior; increased amounts of typing behavior in social apps; increased use of certain punctuation marks, question marks and exclamation marks; faster typing behavior.

7. The method of claim 1, wherein said reference is at least one usage behavior parameter which has been determined in a dataset comprising usage data for a mobile device within a second predefined time window prior to the first predefined time window.

8. The method of claim 7, wherein between the second and the first time windows the subject has received a schizophrenia therapy or a therapy for the negative symptoms associated therewith.

9. The method of claim 8, wherein said therapy is a drug-based therapy.

10. The method of claim 8, wherein an improvement of at least one negative symptom associated with schizophrenia is indicative for a successful therapy.

11. The method of claim 1, wherein said mobile device is a smartphone, smartwatch, wearable sensor, portable multimedia device or tablet computer.

12. The method of claim 1, wherein said subject is a human.

13. A mobile device, comprising:
at least one sensor configured for recording usage data;
a database; and
a processor having stored thereon computer-executable instructions for performing the method according to claim 1.

14. A system comprising the mobile device as recited in claim 13 and a remote device operatively linked to the mobile device.

15. A method of assessing schizophrenia in a subject, comprising:
a) collecting the subject's usage data for a mobile device over a first predefined time window;
b) determining a usage behavior parameter from the usage data;
c) comparing the determined usage behavior parameter to a reference; and
d) determining an improvement, persistency or worsening of negative symptoms associated with schizophrenia in the subject based on the comparison of step (c).

16. The method of claim 15, wherein said reference is a usage behavior parameter which has been determined from usage data from a mobile device within a second predefined time window prior to the first predefined time window.

17. The method of claim 16, comprising administering a schizophrenia therapy between the second predefined time window and the first predefined time window.

18. The method of claim 17, wherein said therapy is a drug-based therapy.

19. The method of claim 18, wherein the drug-based therapy comprises one or more of aripiprazole, asenapine, brexpiprazole, cariprazine, chlorpromazine, fluphenazine, iloperidone, loxapine, lurasidone, molindone, paliperidone, perphenazine, prochlorperazine, risperidone, trifluoperazine, amisulpride, olanzapine, quetiapine, haloperidole, and clozapine.

20. The method of claim 15, wherein the usage data comprises data collected by a plurality of sensors.

21. The method of claim 20, wherein the sensors include one or more of gyroscope, magnetometer, accelerometer, proximity sensors, thermometer, pedometer, fingerprint detectors, touch sensors, voice recorders, light sensors, pressure sensors, location data detectors, cameras, GPS.

22. The method of claim 20, wherein at least one of the sensors is an ambient light sensor and the ambient light data is used in step b) to assess the duration of time the mobile device is in the subject's pocket and/or used in the dark.

23. The method of claim 20, wherein at least one of the sensors is a proximity sensor and the proximity data is used in step b) to assess proximity of objects.

24. The method of claim 15, wherein the usage behavior parameter is one or more of the following combinations of usage behavior parameters:
phone and app usage parameters, ambient sound, movement parameters, and light and proximity parameters;
phone and app usage parameters, movement parameters, and light and proximity parameters;
phone and app usage parameters, ambient sound, and light and proximity parameters;
phone and app usage parameters, ambient sound, and movement parameters;
ambient sound, movement parameters, and light and proximity parameters;
phone and app usage parameters and ambient sound;
phone and app usage parameters, and movement parameters;
phone and app usage parameters, and light and proximity parameters;
ambient sound, and movement parameters; and
ambient sound, and light and proximity parameters.

25. The method of claim 24, wherein the combination of usage behavior parameters further includes a touch behavior parameter.
